(19) United States
(12) Patent Application Publication
(10) Pub. No.: US 2019/0323895 A1
(43) Pub. Date: Oct. 24, 2019

(54) SYSTEM AND METHOD FOR HUMAN TEMPERATURE REGRESSION USING MULTIPLE STRUCTURES

(71) Applicant: Helen of Troy Limited, St. Michael (BB)

(72) Inventors: Theodore Paul Kostopoulos, Hudson, MA (US); James Gorsich, Los Angeles, CA (US)

(21) Appl. No.: 16/390,493

(22) Filed: Apr. 22, 2019

Related U.S. Application Data
(60) Provisional application No. 62/661,671, filed on Apr. 24, 2018.

(51) Int. Cl. G01J 5/00; G01J 5/02; G01J 5/48; A61B 5/01; G06T 7/00; G16H 50/20

(52) U.S. Cl. CPC G01J 5/0025; G01J 5/025; G01J 5/0265; G01J 5/48; G06T 2207/20084; G06T 7/0012; G16H 50/20; G01J 2005/0077; G06T 2207/10048; A61B 5/015

(57) ABSTRACT

A thermal sensing device including a plurality of sensors with at least one of an infrared sensor for capturing infrared data from a biological subject and an imaging sensor for capturing a thermal image of the biological subject. The device includes a display for providing a diagnostic output about the biological subject. Further, a processor operably connected for computer communication with the plurality of sensors and the display identifies at least one feature in the thermal image using a machine learning process, determines the diagnostic output based on the infrared data corresponding to the at least one feature, controls the display to provide the diagnostic output.

### BACKGROUND

Many technologies have been developed to provide indications of various health conditions based on different types of health related data, for example, body temperature. Typically, body temperature is measured at a single area of a biological being, for example, the forehead, the mouth, the ear, the armpit, among others. However, these measurements can include errors based on the sensing technology utilized, the area measured, and other factors specific to a biological being and a surrounding environment. For example, the optimal area for measurement can vary based on the biological being and/or the health related data measured. Further, advances in sensing and computing technology, allow collection of health related data from different sources. Using these advances in sensing and computing technology, measurement errors can be minimized and more in-depth diagnosis of different health conditions can be performed.

### BRIEF DESCRIPTION

According to one aspect, a thermal sensing device includes a plurality of sensors including at least one of an infrared sensor for capturing infrared data from a biological subject and an imaging sensor for capturing a thermal image of the biological subject. The device includes a display for providing a diagnostic output about the biological subject. A processor is operably connected for computer communication with the plurality of sensors and the display. The processor identifies at least one feature in the thermal image using a machine learning process, determines the diagnostic output based on the infrared data corresponding to the at least one feature, and controls the display to provide the diagnostic output.

According to another aspect, a computer-implemented method for thermal sensing of a biological subject includes receiving infrared data about the biological subject from an infrared sensor and a thermal image about the biological subject from an imaging sensor. The method includes identifying at least one feature in the thermal image using a machine learning process, and determining a diagnostic output based on the infrared data corresponding to the at least one feature. The method includes controlling a display of a thermal sensing device to provide the diagnostic output.

According to a further aspect, a device for biological data measurement, includes a plurality of sensors for measuring biometric data about a biological subject and a processor operably connected for computer communication to the plurality of sensors. The processor is operable to receive the biometric data from the plurality of sensors and identify multiple physiological structures of the biological subject. The processor is operable to derive biological data associated with each physiological structure of the multiple physiological structures, and determine a diagnostic output based on the biological data associated with each physiological structure of the multiple physiological structures.

### BRIEF DESCRIPTION OF THE DRAWINGS

The novel features believed to be characteristic of the disclosure are set forth in the appended claims. In the descriptions that follow, like parts are marked throughout the specification and drawings with the same numerals, respectively. The drawing figures are not necessarily drawn to scale and certain figures may be shown in exaggerated or generalized form in the interest of clarity and conciseness. The disclosure itself, however, as well as a preferred mode of use, further objects and advances thereof, will be best understood by reference to the following detailed description of illustrative embodiments when read in conjunction with the accompanying drawings.

FIG. 1 is a block diagram of an illustrative architecture for biological data measurement according to an exemplary embodiment.

FIG. 2 is a schematic view of a thermal sensing device according to an exemplary embodiment.

FIG. 3 is a schematic view of a biological measurement application according to an exemplary embodiment.

FIG. 4 is a process flow diagram of a method for biological data measurement according to an exemplary embodiment.

FIG. 5 is a process flow diagram of a method for identifying areas of interest according to an exemplary embodiment.

FIG. 6A is a schematic diagram of an image of a biological being according to an exemplary embodiment.

FIG. 6B is a schematic diagram of facial feature points of the biological being of FIG. 6A according to an exemplary embodiment.

FIG. 6C is a schematic diagram of facial veins and arteries of the biological being of FIG. 6A according to an exemplary embodiment.

FIG. 7 is an exemplary neural network according to an exemplary embodiment.

FIG. 8 is a process flow diagram of a method for utilizing the neural network of FIG. 7 for determining diagnostic values and identifying health conditions according to an exemplary embodiment.

FIG. 9 is a process flow diagram of a method for training a neural network according to an exemplary embodiment.

FIG. 10 is a neural network architecture according to an exemplary embodiment.

FIG. 11 is a process flow diagram of a method for classifying diagnostic values and identifying health conditions using a machine learning process according to an exemplary embodiment.

FIG. 12 is a process flow diagram of a method for biological data measurement using a thermal sensing device according to an exemplary embodiment.

### DETAILED DESCRIPTION

### I. System Overview

The systems and methods described herein are generally directed to using multiple inputs from sensors associated with multiple identified areas of interests (e.g., physiological structures) of a biological being (e.g., a biological subject) and using the multiple inputs to determine diagnostic information, which is also referred to herein as diagnostic output, including for example, values (e.g., core body temperature) and/or classifications or conditions (e.g., febrile/non-febrile classification, a medical diagnosis). In particular, in some embodiments discussed herein, the multiple inputs include thermal data (e.g., from infrared and/or image sensors) about different physiological structures, which are used to determine a core body temperature of a biological being. In some embodiments, machine learning and deep learning techniques, namely, neural networks, are utilized to determine and/or classify diagnostic values and/or health conditions. For example, regression forms based on multiple physiological structures and neural network modeling can provide outputs with high confidence.

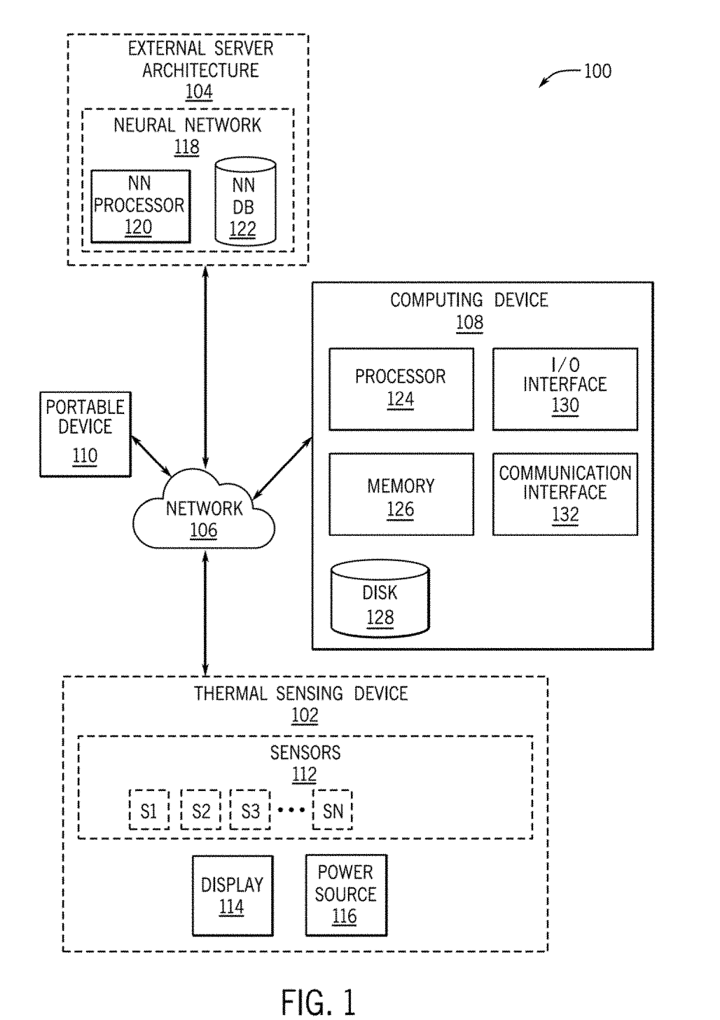

Referring now to the drawings, wherein the showings are for purposes of illustrating one or more exemplary embodiments and not for purposes of limiting the same, FIG. 1 is a block diagram of an architecture 100 for biological data measurement according to an exemplary embodiment. The components of the architecture 100, as well as the components of other systems, hardware architectures, and software architectures discussed herein, may be combined, omitted, or organized into different architectures for various embodiments. FIG. 1 includes a thermal sensing device 102 and an external server architecture 104 operably connected for computer communication via a network 106. FIG. 1 also includes a computing device 108, one or more of the components of which can be implemented with the thermal sensing device 102, the external server architecture 104, and/or the network 106. Additionally, FIG. 1 includes a portable device 110, operably connected for computer communication to the network 106. One or more of the components of the portable device 110 can be implemented with the thermal sensing device 102, the external server architecture 104, and/or the network 106. In some embodiments, the computing device 108 is integrated, in part and/or in whole, with the portable device 110.

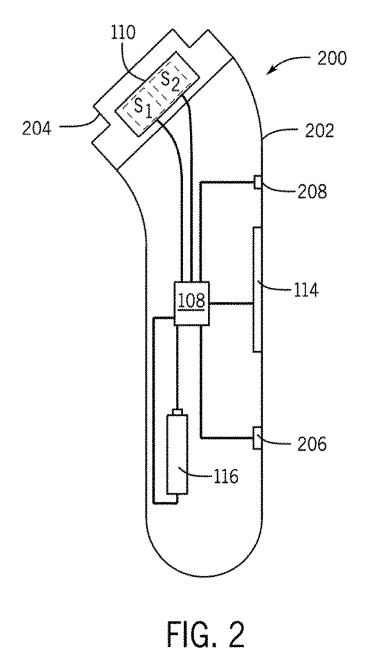

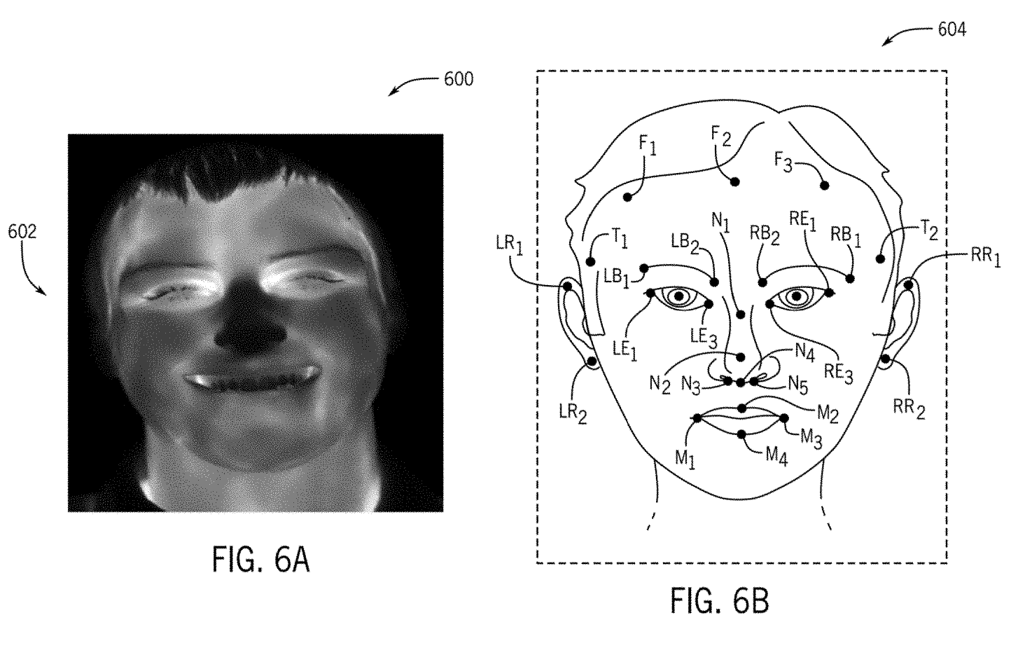

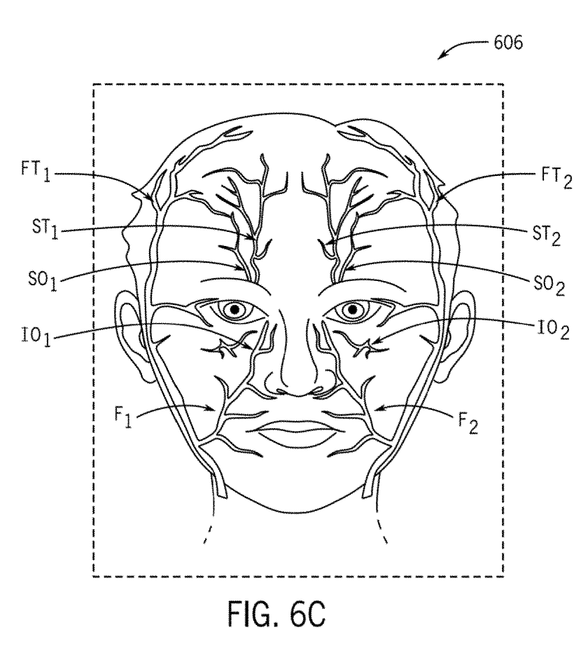

In the exemplary embodiments discussed herein, the thermal sensing device 102 is a thermometry device for detecting body temperature of a biological being (See biological being in FIGS. 6A, 6B, 6C). It is understood that the thermal sensing device 102 can detect body temperature or other biometric data from any area of open skin of the biological being, for example, a forehead, a facial area, a foot, an area of a leg, of the biological being, among others. In some embodiments, the thermal sensing device 102 can be configured as a non-contact thermometer 200 shown in FIG. 2. However, it is understood that the thermal sensing device 102 can be any type of device for acquiring data, temperature data or other biometric data, about the biological being. In FIG. 1, the thermal sensing device 102 includes a plurality of sensors 112 including sensors S₁, S₂, S₃ . . . Sₙ. It is understood that the plurality of sensors 112 can include any number of sensors and these sensors be of different types and configurations. For example, in some embodiments, each of the sensors in the plurality of sensors 112 are non-contact sensors. In other embodiments, the sensors in the plurality of sensors 112 include non-contact sensors and contact sensors. In some embodiments, one or more of the sensors in the plurality of sensors 112 comprise one or more sensor arrays and/or sensor assemblies.

In some embodiments, one or more of the plurality of sensors 112 can be part of a monitoring system (not shown) that provides monitoring information (e.g., biometric data) about the biological being. In some embodiments, one or more of the sensors of the plurality of sensors 112 can be integrated with the portable device 110, which can be, for example, a medical device, a wearable device, or a smart phone associated with the biological being. For example, one or more of the sensors of the plurality of sensors 112 can be integrated with the portable device 110. Thus, one or more of the plurality of sensors 112 can be physically independent from the thermal sensing device 102, and measurements from these sensors can be communicated to the thermal sensing device 102 and/or the computing device 108 via the network 106 and/or other wired or wireless communication protocols.

Generally, the plurality of sensors 112 monitor and provide biometric information related to the biological being. The biometric information includes information about the body of the biological being that can be derived intrinsically or extrinsically. Biometric information can include, but is not limited to, thermal data (e.g., temperature data, thermograms), heart rate, blood pressure, blood flow, photoplethysmogram, oxygen content, blood alcohol content (BAC), respiratory rate, perspiration rate, skin conductance, pupil dilation information, brain wave activity, digestion information, salivation information, eye movements, mouth movements, facial movements, head movements, body movements, hand postures, hand placement, body posture, among others.

As mentioned above, the plurality of sensors 112 can include sensors that measure information using different types of technologies. For example, one or more of the plurality of sensors 112 can include electric current/potential sensors (e.g., proximity, inductive, capacitive, electrostatic), acoustic sensors, subsonic, sonic, and ultrasonic sensors, vibration sensors (e.g., piezoelectric), optical sensors, imaging sensors, thermal sensors, temperature sensors, pressure sensors, photoelectric sensors, among others. Further, in some embodiments, one or more of the plurality of sensors 112 can be sensors that measure a specific type of information using different sensing technologies. For example, one or more of the plurality of sensors 112 can be heart rate sensors, blood pressure sensors, oxygen content sensors, blood alcohol content (BAC) sensors, electroencephalogram (EEG) sensors, functional near infrared spectroscopy (FNIRS) sensors, functional magnetic resonance imaging (FMRI) sensors, among others.

In one embodiment, the plurality of sensors 112 includes an infrared (IR) sensor, an imaging sensor, and/or a conduction sensor. An IR sensor can include a thermopile or transducer. The IR sensor can detect infrared radiation (e.g., infrared data) and output a voltage signal corresponding to the detected radiation. The voltage signal can then be converted into a measured (e.g., temperature) value. In some embodiments, the temperature value is an average temperature detected within the field of view of the IR sensor. In the embodiments discussed herein, the IR sensor is a non-contact sensor. For simplicity, with respect to the exemplary embodiments discussed herein and FIG. 1, the IR sensor will be referred to as the IR sensor S₁.

In one embodiment, the plurality of sensors 112 includes an imaging sensor. In some embodiments, the imaging sensor is a digital camera or digital video camera, for example, having a complementary metal-oxide-semiconductor (CMOS), a charge-coupled device (CCD) or a hybrid semiconductor imaging technology. The imaging sensor can be capable of high definition imaging or video capture with a wide-angle capture. In some embodiments, the imaging sensor is a thermographic camera that can detect radiation in the long-infrared range of the electromagnetic spectrum (roughly 9,000-14,000 nanometers or 9-14 μm) and produce images of that radiation (e.g., thermograms, thermal image). In other embodiments, the imaging sensor is capable of detecting visible light and can produce digital images and/or video recordings of the visible light. For example, in some embodiments, the imaging sensor can be used to visualize the flow of blood and small motions from the biological being. This information can be used to detect heart rate, pulse, blood flow, skin color, pupil dilation, respiratory rate, oxygen content, blood alcohol content (BAC), among others. For simplicity, with respect to the exemplary embodiments discussed herein and FIG. 1, the imaging sensor will be referred to as the imaging sensor S₂.

As mentioned above, in one embodiment, the plurality of sensors 112 can include a conduction sensor. The conduction sensor can be a contact-type sensor that relies upon conduction between the biological being and a component of the conduction sensor, for example, a thermistor. The contact causes heating of the sensor component which is detected and converted into a value, for example, a corresponding temperature of the biological being. The thermistors are also used to measure the ambient temperature. For simplicity, with respect to the exemplary embodiments discussed herein and FIG. 1, the conduction sensor will be referred to as the conduction sensor S₃.

Referring again to FIG. 1, the thermal sensing device 102 can also include a display 114 and a power source 116. The display 114 can function as an input device and/or an output device. For example, the display 114 can be a display screen (e.g., LCD, LED) that can output information such as readings from the plurality of sensors 112 or other diagnostic values and/or health conditions as discussed herein. In one embodiment, the display 114 can output a thermal image of the biological being or a thermal image of a part of the biological being. For example, the display 114 can output the exemplary image 600 shown in FIG. 6A. In some embodiments, more than one thermal image can be output to the display 114. Power can be provided to the thermal sensing device 102 by the power source 116 which can comprise disposable batteries, rechargeable batteries, and capacitive storage, among others. In some embodiments, the power source 116 can also include a power jack for connection of a power cord to a wall outlet, USB outlet, or other charging port.

As mentioned above, an exemplary thermal sensing device is shown as the non-contact thermometer 200 in FIG. 2. Here, the non-contact thermometer 200 includes a housing 202, which can be held by a user's hand (not shown). However, in other embodiments, the housing 202 could be attached to a movable or stationary unit (not shown) for monitoring. For example, the housing 202 can be adapted to attach the non-contact thermometer 200 so that the non-contact thermometer hangs above a hospital bed to monitor a patient in the hospital bed. A probe 204 can extend from the housing 202. In some embodiments, the probe 204 is placed in proximity to the skin of the biological being and a measurement is captured. A switch 206 can be used to power up the non-contact thermometer 200 using the power source 116, and an optional switch 208 can be used to initiate a measurement reading. The measurement (e.g., biometric data, a core body temperature, a thermal image) can be displayed on the display 114. The display 114, the power source 116, the switch 206, the switch 208 are all operably connected for computer communication with a controller, for example, in FIG. 2, the computing device 108. In FIG. 2, the non-contact thermometer 200 includes the sensors 112, namely, the IR sensor S₁ and the imaging sensor S₂. However, it is appreciated that in other embodiments, different numbers of sensors and different types of sensors can be implemented. As shown in FIG. 2, the sensors 112 are also operably connected for computer communication with the computing device 108.

Referring again to FIG. 1, the architecture 100 can include the external server architecture 104, which can be implemented using a centralized, a distributed, and/or a cloud computing system architecture. As shown in FIG. 1, the external server architecture 104 can host a neural network 118, which includes a neural network processor 120 and a neural network database 122. The neural network database 122 can include classification data and classification models related to biometric data and health conditions. The classification data and classification models can be based on population data and/or data collected from specific biological beings. In some configurations, in addition to being hosted on the external server architecture 104, the neural network 118 or specific subsets (not shown) of the neural network 118 can be hosted and/or executed by the thermal sensing device 102, the computing device 108 and/or the portable device 110. Thus, one or more of the components of the neural network 118 could be implemented locally with the thermal sensing device 102, the computing device 108, and/or the portable device 110.

As will be discussed herein, the neural network 118 is utilized and/or trained to identify areas of interest and/or physiological structures of the biological being, perform regression analysis of the biometric data to determine diagnostic values (e.g., core body temperature), and/or perform classification of the biometric data to identify health conditions (e.g., febrile/non-febrile). In some embodiments, the external server architecture 104 can host one or more machine learning processors (e.g., neural network processor 120) that may execute various types of machine learning methods (e.g., models, algorithms). For example, in some embodiments, the neural network 118 includes pre-trained models that are utilized for identifying areas of interest, regression analysis, and/or classification. In other embodiments, the methods and systems discussed herein can apply to the training of the neural network 118 and creation of machine learning models and algorithms.

Referring again to FIG. 1, the computing device 108 can include provisions for processing, communicating and interacting with various components of the thermal sensing device 102, the external server architecture 104, the network 106, the portable device 110, and other components of the architecture 100. The computing device 108 can be implemented in whole or in part by any combination of the thermal sensing device 102, the external server architecture 104, the network 106, and/or the portable device 10. Thus, the computing device 108 can be a standalone remote device that receives biometric data from the plurality of sensors 112 and executes the processing and analysis of the biometric data. In other embodiments, the computing device 108 is integrated with the thermal sensing device 102, for example, as shown in FIG. 2. It is understood that other implementations of one or more components of the computing device 108 can be considered.

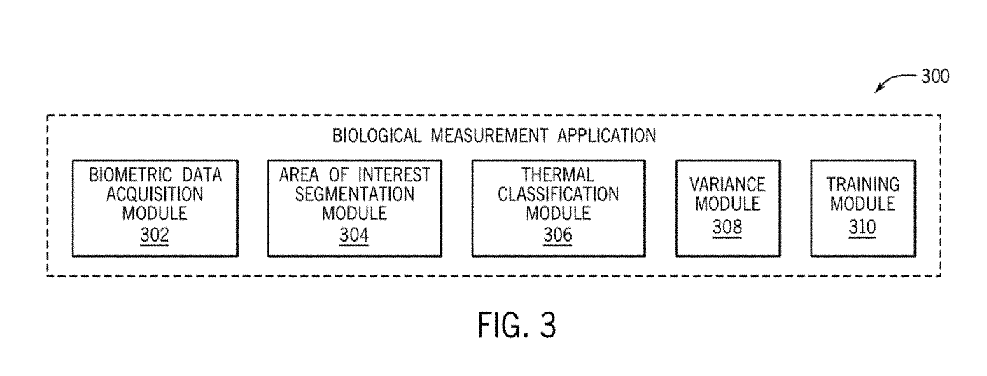

The computing device 108 will now be described in more detail with reference to FIG. 1. In FIG. 1, the computing device 108 includes a processor 124, a memory 126, a data store (e.g., disk) 128, an input/output (I/O) interface 130, and a communication interface 132, each of which can be operably connected for computer communication using any wired and/or wireless hardware or protocols, for example, a bus (not shown). The processor 124 can include a graphics processing unit (GPU), logic circuitry (not shown) with hardware, firmware, and software architecture frameworks for facilitating biometric data measurement and processing with the components of the computing device 108, the thermal sensing device 102, the external server architecture 104, the portable device 110, and other components of the architecture 100. Thus, in some embodiments, the processor 124 can store application frameworks, kernels, libraries, drivers, application program interfaces, among others, to execute and control hardware and functions discussed herein. For example, the processor 124 can include various modules which will be described herein with FIG. 3. In some embodiments, the memory 126 and/or the data store 128 can store similar components as the processor 124 for execution by the processor 124.

The I/O interface 130 can include one or more input/output devices including software and hardware to facilitate data input and output between the components of the computing device 108 and other components, networks, and data sources, of the architecture 100 and the non-contact thermometer 200. For example, the I/O interface 130 can include input components (e.g., touch screen, buttons) for receiving user input. The I/O interface 130 can also include the display 114, or other visual, audible, or tactile components for providing input and/or output. Further, in some embodiments, the I/O interface 130 can include the plurality of sensors 112 to provide input in the form of the biometric data measured by the plurality of sensors 112.

With respect to the plurality of sensors 112, signals output from the plurality of sensors 112 can be received and/or acquired by the processor 124 (e.g., via the I/O interface 130) and can be stored at the memory 126 and/or the data store 128. In some embodiments, the processor 124 can process signal output by sensors and devices into data formats that include values and levels. Such values and levels can include, but are not limited to, a numerical or other kind of value or level such as a percentage, a non-numerical value, a discrete state, a discrete value, a continuous value, among others. For example, in some cases, the value or level of X can be provided as a percentage between 0% and 100%. In other cases, the value or level of X can provided as a value in the range between 1 and 10. In still other cases, the value or level of X may be a temperature measurement. In still other cases, the value or level of X may not be a numerical value, but could be associated with a determined state or classification, such a health state or a health condition.

Referring again to FIG. 1, the communication interface 132 can include software and hardware to facilitate data communication between the components of the computing device 108 and other components of the architecture 100. Thus, the communication interface 132 can provide wired and/or wireless communications with external networks or devices. More specifically, the communication interface 132 can include network interface controllers (not shown) and other hardware and software that manages and/or monitors connections and controls bi-directional data transfer between the communication interface 132, components of the computing device 108, components of the thermal sensing device 102, and/or components of the external server architecture 104. Accordingly, the communication interface 132 can provide wired or wireless computer communications utilizing various protocols to send/receive non-transitory signals internally to the plurality of components of the computing device 108 and/or externally (e.g., via the network 106) to the thermal sensing device 102 and/or the external server architecture 104. Generally, these protocols can include a wireless system (e.g., IEEE 802.11 (WiFi), IEEE 802.15.1 (Bluetooth®)), a near field communication system (NFC) (e.g., ISO 13157), a local area network (LAN), a cellular network, and/or a point-to-point system.

As mentioned above, the processor 124 can implement and can include various applications, modules, and/or instructions for biological data measurement and processing. For example, with reference to FIG. 3, a biological measurement application 300 according to an exemplary embodiment is shown. The biological measurement application 300 includes a biometric data acquisition module 302, an area of interest segmentation module 304, a thermal classification module 306, a variance module 308, and a training module 310, each of which will be described in further detail herein. More specifically, the processor 124 can use and/or execute the biological measurement application 300 to apply the neural network 118 and/or train the neural network 118 based on multiple inputs from the plurality of sensors 112 associated with multiple areas of interest of the biological being, the result of which can provide diagnostic information, for example, core body temperature and/or health condition classification.

### II. Methods for Biological Data Measurement and Processing

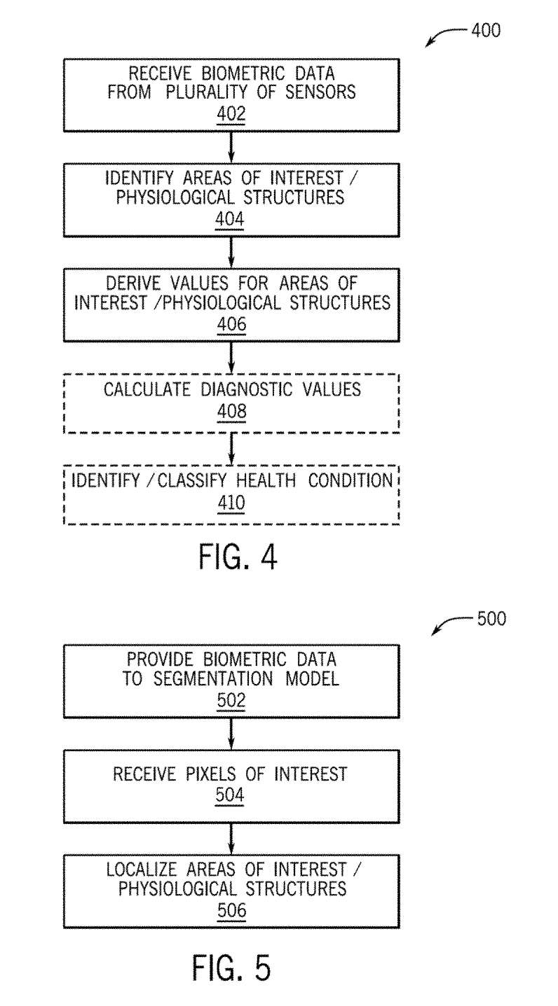

Exemplary methods for biological data measurement and processing will now be described in detail in conjunction with the components of FIGS. 1, 2, and 3. Referring now to FIG. 4, a method 400 for biological data measurement is shown. At block 402, the method 400 includes receiving biometric data from one or more of the plurality of sensors 112. For example, the biometric data acquisition module 302 executed by the processor 124 can receive the biometric data (e.g., infrared data, thermal images) from the plurality of sensors 112. In some embodiments, after receiving the biometric data, the biometric data acquisition module 302 can store the biometric data for example, at the memory 126 and/or the disk 128.

In some embodiments, the biometric data acquisition module 302 can receive other data related to the biometric data, for example, characteristics that describe the biometric data. For example, a time associated with the measurement of the biometric data, a device (e.g., the portable device 110) that measured the biometric data, an identified sensor that measured the biometric data, a type of sensor (e.g., sensing technology) that measured the biometric data, the type of biometric data (e.g., heart rate data, temperature data), among others. In some embodiments, in addition to the biometric data, other types of data about the biological being can be received. For example, demographic information, an identity of the biological being, among others. The demographic information can be received for example via input to the thermal sensing device 102 and/or received as stored data from, for example, the memory 126 and/or the disk 128.

As discussed above with FIG. 1, different types of biometric data can be measured from the plurality of sensors 112. For example, in one embodiment, at block 402, the processor 124 receives non-contact thermal data from the IR sensor S₁ and thermal data in the form of images from the imaging sensor S₂. In other embodiments, contact thermal data can also be received from the conduction sensor S₃. As mentioned above, in some embodiments, a location associated with the biometric data (e.g., an area of the biological being where the measurement of the biometric data was retrieved) can be received by the biometric data acquisition module 302. As an illustrative example, non-contact thermal data from the IR sensor S₁ can be associated with a location on a forehead of the biological being or a foot of the biological being. As discussed above, the location can be stored in association with the biometric data at the memory 126 and/or the disk 128.

At block 404, the method 400 includes identifying areas of interests of the biological being based on the biometric data. In some embodiments, identifying the areas of interest includes identifying multiple physiological structures of the biological being. An area of interest can be a region of the biological being for example a defined surface area of the biological being. A physiological structure is a defined anatomical part of the biological being, for example, a nose, a forehead, a chin, a foot, one or more facial feature points, one or more blood vessels, among others. In the embodiments discussed herein, the area of interest segmentation module 304 executed by the processor 124 can identify the areas of interest by classifying the biometric data. Block 404 will now be described in more detail with respect to FIG. 5.

### A. Identifying Areas of Interests/Multiple Physiological Structures

Referring now to FIG. 5, at block 502, the method 500 includes providing biometric data to a segmentation model. For example, the area of interest segmentation module 304 can provide the biometric data to the neural network 118 and/or apply a segmentation model of the neural network 118 to the biometric data. The segmentation model is generated by machine learning techniques that given an input (e.g., biometric data) is trained by example inputs to output information about areas of interest and/or physiological structures that are most impactful for classifying a health condition and/or determining a diagnostic value. For example, with respect to core body temperature, the segmentation model 304 may output information related to multiple facial areas or facial structures that are optimal for determining a core body temperature and/or identifying a health condition. In some embodiments, a pre-trained segmentation model stored at the neural network 118 is applied to the biometric data. The area of interest segmentation module 304 can output the areas of interest (e.g., pixels of interest, locations of pixels, facial coordinates) based on the biometric data and the pre-trained segmentation model.

As an illustrative example, one or more images (e.g., from the imaging sensor S₂) representing biometric data can be received by the biometric data acquisition module 302. For example, the imaging sensor S₂ can capture images of the head of the biological being including the face of the biological being. FIG. 6A illustrates an exemplary image 600 (e.g., acquired by the imaging sensor S₂) of a biological being 602. Here, the image 600 is a thermogram including the head of the biological being 602 according to a frontal view of the face of the biological being. The gradient variations shown in the image 600 correspond to variations in temperature as emitted amounts of radiation. The image 600 has been preprocessed and it is understood that any type of image pre-processing (e.g., filters, conversion to gray scale) can be implemented.

The images can be trained against the pre-trained segmentation model. For example, the pre-trained segmentation model can output pixels that indicate areas that are most impactful for deriving diagnostic information (e.g., core body temperature) on the biological being. Accordingly, at block 502, the pre-trained segmentation model is applied to the image 600, using for example, class activation maps as is known with convolution neural networks. Pixels are tagged if, for example, the intensity of the pixels is above a predetermined threshold. Accordingly, at block 504, the method 500 includes receiving pixels (e.g., pixels of interest, locations of pixels, facial coordinates) of interest as output from the pre-trained segmentation model. From the identified pixels and the biometric data, areas of interest (e.g., physiological structures, feature points) of the biological being can be identified for further processing.

More specifically, at block 506, the method 500 includes localizing the areas of interests and/or the physiological structures based on the output from the segmentation model. For example, the images captured from the imaging sensor S₂ can be processed for feature extraction and/or facial recognition by the area of interest segmentation module 304. In particular, based on the identified pixels a plurality of facial feature points can be extracted from the images corresponding to the areas of interest and/or physiological structures. Known feature extraction and/or recognition techniques can be used to process the image 600 and extract the plurality of facial feature points from the images. For example, the plurality of facial feature points can be extracted from the images by searching for feature points based on face geometry algorithms and matching using the pixels identified at blocks 502 and 504. Feature points can be of different types, for example, region, landmark, and contour. FIG. 6B illustrates a schematic view 604 of the image 600 including exemplary facial feature points. The exemplary facial feature points are described in Table 1. It is understood that the facial feature points are exemplary in nature and that other facial feature points or body feature points (e.g., foot, arm, leg) can be implemented. For example, in some embodiments, one or more blood vessels and/or blood vessel networks (e.g., temporal artery, supraorbital artery, infraorbital artery, carotid artery) can be identified as an area of interest and/or physiological structure. FIG. 6C illustrates another schematic view 606 of the image 600 including exemplary facial feature points of facial veins and/or arteries. These exemplary facial feature points are also described in Table 1.

TABLE 1

| Reference Number | Facial Feature Points |
|---|---|
| LB₁ | Outer corner of left eyebrow |
| LB₂ | Inner corner of left eyebrow |
| RB₁ | Outer corner of right eyebrow |
| RB₂ | Inner corner of right eyebrow |
| LE₁ | Outer corner of left eye |
| LE₃ | Inner corner of left eye |
| RE₁ | Outer corner of right eye |
| RE₃ | Inner corner of right eye |
| N₁ | Top of nose bridge |
| N₂ | Nose tip |
| N₃ | Left Naris (left corner of left nostril) |
| N₄ | Columella (connecting tip of nose to base of nose) |
| N₅ | Right Naris (right corner of right nostril) |
| M₁ | Left mouth corner |
| M₂ | Cupid's bow (top of mouth) |
| M₃ | Right mouth corner |
| M₄ | Bottom of mouth |
| F₁ | Left area of forehead |
| F₂ | Center area of forehead |
| F₃ | Right area of forehead |
| T₁ | Left temple |
| T₂ | Right temple |
| LR₁ | Left ear helix |
| LR₂ | Left ear lobe |
| RR₁ | Right ear helix |
| RR₂ | Right ear lobe |
| T₁ | Left superficial temporal |
| T₂ | Right superficial temporal |
| ST₁ | Left supratrochlear |
| ST₂ | Right supratrochlear |
| SO₁ | Left supraorbital |
| SO₂ | Right supraorbital |
| IO₁ | Left infraorbital |
| IO₂ | Right infraorbital |
| F₁ | Left facial |
| F₂ | Right facial |

It is also understood that localizing the areas of interests and/or the physiological structures can include more than one facial feature points. For example, based on the pixels identified by the pre-trained neural network, the processor 124 can identify multiple areas of interests and/or multiple physiological structures by locating the pixels and graduating the area in proximity to the pixels. As an illustrative example with respect to FIG. 6B, one or more pixels may be identified that correspond to facial features N₁, N₂, LR₁, and LR₂. The processor 124 can identify these facial features based on the pixels and localize the areas of interests and/or the physiological structures. For example, the nose can be identified as an area of interest and/or physiological structure and graduated to include areas surrounding the facial features N₁, N₂. As another example, the left ear can be identified as an area of interest and/or physiological structure and graduated to include areas surrounding the facial features LR₁, and LR₂.

As a further example, and as mentioned above with FIG. 6C, locations of blood vessels beneath the skin of the face can be identified as an area of interest and/or physiological structure. For example, one or more pixels may be identified that correspond to facial features SO₁ and IO₁, The processor 124 can identify these facial features based on the pixels and localize the areas of interests and/or the physiological structures. In further embodiments, locations of blood vessels and facial features can be identified as an area of interest and/or a physiological structure. For example, one or more pixels may be identified that correspond to facial features N₁, N₂, LR₁, LR₂, SO₁ and IO₁.

Referring again to FIG. 4, at block 406 the method 400 includes deriving biological values for the areas of interest and/or the multiple physiological structures identified at block 404. Said differently, each area of interest identified at block 404 is analyzed to determine a value of the area of interest. In the embodiments discussed herein, the thermal classification module 306 executed by the processor 124 can derive biological values for the areas of interest and/or the multiple physiological structures identified at block 404. In some embodiments, the biological value can be a temperature or a thermal gradient of the area of interests and/or multiple physiological structures. In other embodiments, the biological value can be a heart rate, blood pressure value, among others. In some embodiments the biological value is retrieved and or determined based on stored biometric data associated with the area of interests and/or multiple physiological structures. Based on the biological values derived from the multiple areas of interest and/or multiple physiological structures, diagnostic information can be calculated and/or classified, for example, using a neural network for regression and/or classification. For example, at block 408, the method 400 can include calculating diagnostic values based on the biological values for the areas of interest and/or the multiple physiological structures determined at block 406. At block 410, the method 400 can further include identifying and/or classifying a health condition based on the biological values for the areas of interest and/or the multiple physiological structures determined at block 406 and/or the diagnostic values determined at block 408. Blocks 408 and 410 related to embodiments for determining diagnostic values (e.g., core body temperature) and health conditions (e.g., febrile/non-febrile, medical diagnosis) will now be discussed in further detail.

### B. Neural Network for Regression and Classification of Biological Data

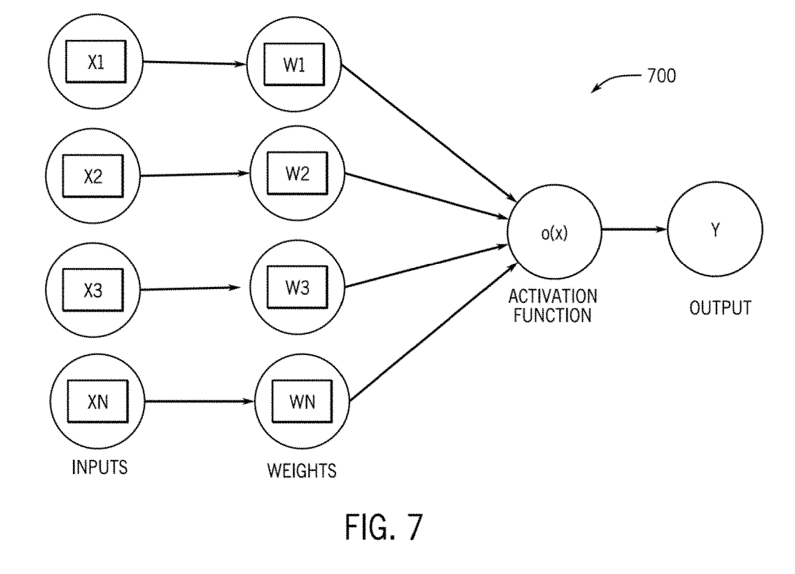

As mentioned above, diagnostic values and/or health conditions can be determined using machine learning algorithms and neural networks. An exemplary neural network 700 is shown in FIG. 7. The exemplary neural network 700 is a neural network that can be used with regression for determining a continuous value (e.g., a diagnostic value, core body temperature) and/or for classification (e.g., a health condition, febrile/non-febrile). In some embodiments, the neural network 700 is a recurrent convolution neural network (RCNN) for logistic regression and binary classification, however, it is understood that any type of neural network (e.g., CNN) and machine learning algorithms can be implemented. Further, for purposes of the systems and methods discussed herein, the neural network 700 shown in FIG. 7 is simplified and it is understood that the neural network 700 can include any number of layers, nodes, weights, biases, and other components not shown.

In FIG. 7, the neural network 700 includes inputs X1, X2, X3 . . . XN and weights W1, W2, W3 . . . WN. Each input feature X represents a biometric value derived from the identified areas of interests and/or physiological structures as determined at block 406. Thus, the inputs include biometric values from multiple different areas of interest and/or physiological structures. Each input feature X is passed forward and multiplied with a corresponding weight W. The sum of those products can be added to a bias (not shown) and fed to an activation function 6(x) which results into an output \dot{Y}. Thus, the activation function uses multiple areas of interest and/or multiple physiological structures for regression of various biometric values to determine a diagnostic value, for example, core body temperature. In some embodiments, the activation function can be a linear function, a step function, a hyperbolic function, or a rectified linear unit (ReLU), however, it is understood that other types of activation functions can be implemented.

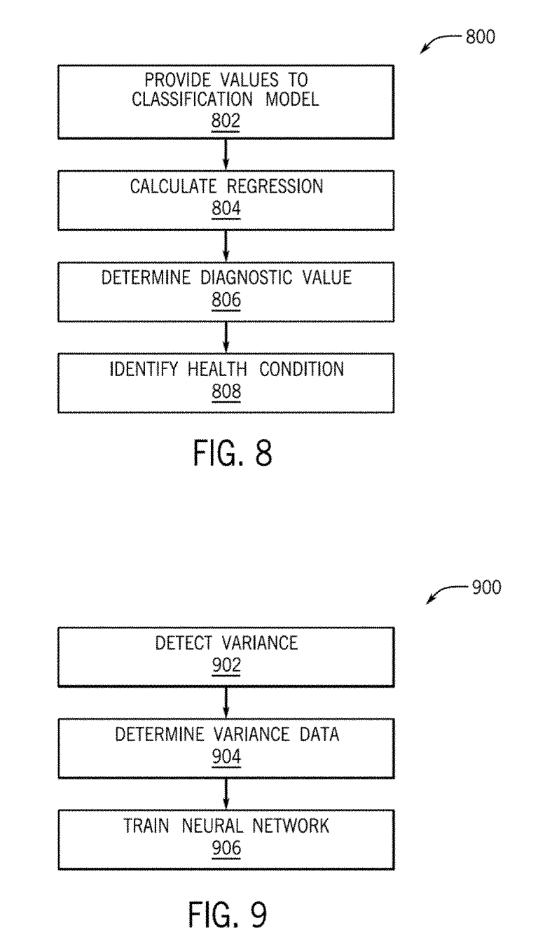

Application of the neural network 700 will now be discussed in detail with the method 800 of FIG. 8. At block 802, the method 800 includes providing the biometric values to a segmentation model. For example, the thermal classification module 306 can provide the biometric values to a segmentation model applied by the neural network 118. As an illustrative example, X1 can be a temperature value or a thermal gradient derived from an image associated with the nose of the biological being, X2 can be a temperature value or a thermal gradient derived from an image and associated with the forehead of the biological being, and X3 can be a temperature value or a thermal gradient derived from an image associated with the ear of the biological being. It is understood that in some embodiments, the inputs can be non-image or thermal gradient biological values. For example, the inputs could include a temperature value based on infrared radiation (e.g., from the IR sensor S₁) associated with one or more of the areas of interest and/or physiological structures, or a heart rate value based on pulse oximetry associated with one or more of the areas of interest and/or physiological structures.

At block 804, the method 800 includes calculating a regression form. More specifically, and as discussed above, multiple areas of interest and/or multiple physiological structures for regression can be performed according to the activation function of the neural network 700. Referring to the illustrative example above, the regression of inputs includes the temperature value or a thermal gradient derived from an image associated with the nose, the temperature value or a thermal gradient derived from an image and associated with the forehead, and the temperature value or a thermal gradient derived from an image and associated with the ear are analyzed according to the activation function. Accordingly, at block 806, the method 800 includes determining a diagnostic value or other health related value based on the biometric values and regression modeling. Thus, in this example, the regression according to the activation function results in a prediction of a continuous diagnostic value, namely, a core body temperature.

Further, in some embodiments, a health condition can be identified based on the inputs applied to the neural network 700 using classification techniques. Thus, at block 808, the method 800 includes identifying a health condition. More specifically, given the inputs discussed above at block 802 and according to the classification model of the neural network 700, the neural network 700 can predict a health condition and/or a health state. For example, a probability that the biological being is healthy or sick. As another example, the prediction can be a particular health aliment or medical diagnosis, for example, febrile, non-febrile, cancer, high blood pressure, heart disease, diabetes, among others. Accordingly, using data from multiple areas of interest allows for correlation and classification to determine diagnostic values and/or identify patterns associated with health conditions.

In some embodiments, the diagnostic value and/or the health condition can be output by the thermal sensing device 102 and/or the computing device 108. For example, the processor 124 can control the display 114 to provide a visual indication of the diagnostic value and/or the health condition. In some embodiments, the diagnostic value and/or the health condition is indicated by a textual description provided by the display 114, a color of the textual description, or a color emitted from the thermal sensing device 102. In other embodiments, an audible indication and/or alert can be provided. The indication of the diagnostic value and/or the health condition can vary as function of the diagnostic value and/or the health condition. Further, it is understood that other types of feedback (e.g., visual, audible, tactile) to provide the resulting information to an end-user of the thermal sensing device 102 can be implemented. For example, with respect to the non-contact thermometer 200, the exemplary thermal image 600 can be output to the display 114.

### C. Neural Network Training for Continuous Learning of Biological Data

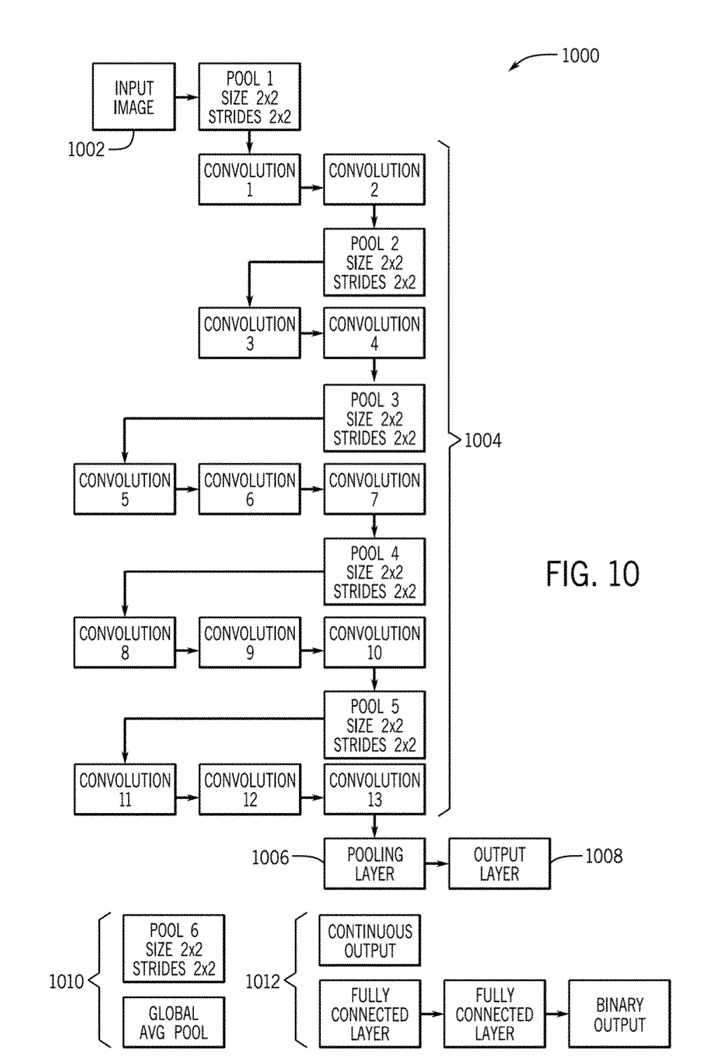

As mentioned above, the methods and systems discussed herein can apply to the training of the neural network 118 and creation of machine learning models and algorithms. In some embodiments, the neural network 118 (e.g., the neural network 700) can be trained to allow for continuous learning and application of biological data. Thus, in some embodiments, the thermal sensing device 102 and/or the computing device 108 can increase the accuracy of predictions over time using the machine learning and neural network techniques described above. Referring now to FIG. 9, a method 900 for training the neural network 700 of FIG. 7 and/or other neural networks discussed herein (e.g., the neural network 1000 of FIG. 10) according to an exemplary embodiment is shown.

At block 902, the method 900 includes detecting a variance. For example, the variance module 308 executed by the processor 124 can detect a variance based on the output of the neural network 700 compared to a target output and/or a ground truth value. For example, a target output can be retrieved from the neural network database 122. With respect to core body temperature, an oral equivalent temperature can be considered the target output. In one embodiment, the oral equivalent temperature can be obtained from the neural network database 122. In other embodiments, the oral equivalent temperature can be biometric data sensed by the plurality of sensors 112, for example, the conduction sensor S₃. As discussed above, the biometric data from the conduction sensor S₃ can be stored at, for example, the memory 126 and/or the disk 128. The variance module 308 can retrieve the biometric data and compare it to the output of the neural network 700. In some embodiments, a variance is detected if the comparison between the output and the target output is within a predetermined threshold.

If a variance is detected at block 902, at block 904, the method 900 includes determining variance data. For example, the variance module 308 executed by the processor 124 can determine the variance data based on the variance detected at block 902. Variance data can include determining a local error and/or a total error for one or more nodes of the neural network 700. Based on the variance data, at block 906, the method 900 includes training the neural network 700. For example, the training module 310 executed by the processor 124 can train the neural network 700 by updating the weights according to the variance data. Thus, the thermal sensing device 102 via the neural network 700 can learn and adapt according to the individual biological being. In some embodiments, this learning mechanism can be used for device calibration, for example, calibration of one or more of the plurality of sensors 112. Accordingly, using multiple physiological structures for biometric data measurement and applying neural network modeling, diagnostic information and health conditions can be predicted with high confidence.

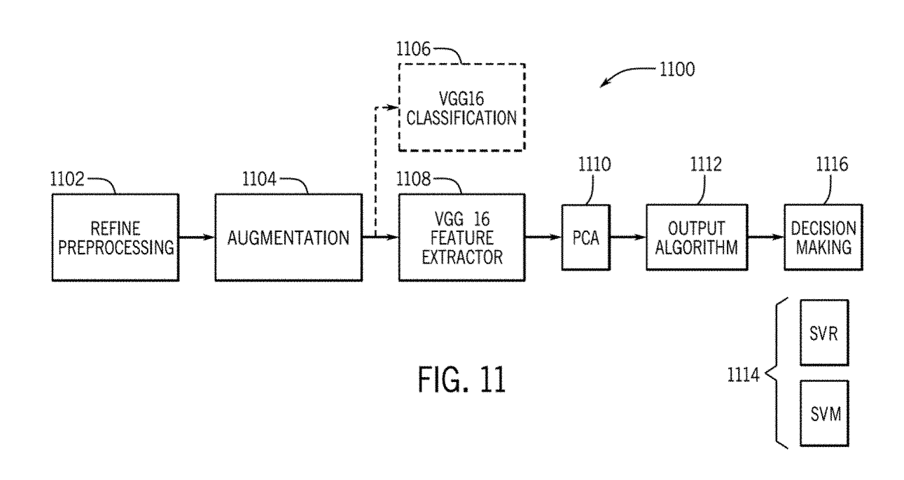

More particular examples of a neural network for determining diagnostic values and/or a classification will now be discussed with reference to FIGS. 10 and 11. An exemplary modified VGG16 neural network architecture 1000 is shown in FIG. 10 and a method 1100 for implementing the neural network architecture 1000 is shown in FIG. 11. It is understood that the neural network architecture 1000 and the method 1100 can be used for training the neural network architecture 1000 and/or for determining a diagnostic value or a health condition. With respect to FIG. 10, an input image 1002 is fed into multiple convoluted neural network layers indicated by element 1004 for feature extraction. The output feature extraction data is then forwarded to a pooling layer 1006. The pooling layer 1006 reduces the dimensionality of the input image 1002. In this example, the pooling layer 1006 can take the form of a Pool 6 layer and/or a global average pool as shown by layers 1010. In one embodiment, the pooling layer selected is based on the type of output desired, continuous or binary. For example, if a continuous output is desired, the dimensionality reduction may be performed using pool 6 and if a binary output is desired, the dimensionality reduction may be performed within the global average pool, or vice versa. The reduced input image 1002 are then fed to the output layer 1008. Here, the output may take the form of a continuous output (e.g., core body temperature, diagnostic value) or a binary output (e.g., febrile/non-febrile, medical diagnosis). With respect to the binary output, two fully connected layers can be implemented.

Referring now to FIG. 11, at block 1102 and block 1104 of the method 1100, image pre-processing and augmentation is performed, for example with the input image 1002. More specifically, input thermal images (e.g., the input image 1002) include facial images of febrile and afebrile biological subjects in which both eyes were present at various distances, facial orientations, emotional statuses (e.g., a baby crying) and acclimation times. The input thermal images are cropped into predetermined pixel sized images and then augmented by rotating each image, then flipping the image along the y-axis, and rotating again along the y-axis, thus providing a plurality of augmented images from a single input thermal image.

Further, each pixel of the input images prior to segmentation was raised to a predetermined power, then normalized to a predetermined maximum value, (e.g., the maximum value being 255), and all other points being their square relation to the predetermined maximum value. The theory behind this preprocessing technique is that due to the high precision of the measurement equipment (i.e., the imaging sensor S₂ used to capture the images), and the ambient temperatures found in an investigation being lower than the physiological temperature present, the assumption can be made that the warmest regions in the image were physiological structures and of interest in the classification.

As mentioned above with FIG. 6C, in one example, the sources of heat are the blood vessels and inner cantus. Squaring these points (e.g., pixels) “pushed” the warmest regions “forward,” giving them more weight for the machine learning algorithm. It was empirically found that higher orders of magnitude (i.e. cubing or putting the data to the 5th power) had a detrimental effect on the results. It is hypothesized this is because it pushes the secondary sources of heat further back, giving all additional weight to the warmest feature or draws too much attention for too few pixels in the area of interest, e.g., only a part of the warmest region could be used due to a large discrepancy in the feature's thermal characteristic.

In FIG. 10, the neural network architecture 1000 was used to identify important nonlinear relationships in the input images. For example, the layers indicated by element 1004 in FIG. 10 represent feature extraction of data. In FIG. 11, at block 1108 feature extraction is performed using, for example, the layers 1004, however, VGG16 classification at block 1106 is optional. Using the end output of the neural network architecture 1000, there would be various defined blocks of data that simplify nonlinear patterns intrinsic to the input images. To simplify the convolutional neural network into its core components, there are many different pixels in the input images. These pixels get passed through many filters that output many different small images to simplify nonlinear trends in the data. These filters are represented by the layers 1004.

Principle Components Analysis (PCA) at block 1110 can be made with the aid of an output algorithm 1112, which can be a Support Vector Machine (SVM) and/or a Support Vector Regression (SVR) as shown by element 1114. PCA with the aid of SVM was found useful to make a classification, e.g. a febrile or non-febrile classification. PCA with the aid of SVR was found useful to make a continuous output (e.g., a core body temperature, a diagnostic value). Principle component analysis (PCA) is a technique used to reduce a large amount of data points of a common feature to a single point. PCA finds similar clusters of data points outputted by a feature extractor utilizing orthogonal vectors. This multitude of data points can be reduced down to a handful of meaningful points to be fed into the SVM. The SVM takes the data fed from the PCA and considers the output a part of hyperplane, a multidimensional plane, to simplify the data to a single classification entity. The algorithm does this by applying vectors to separate the data. The algorithm optimizes the placement in the hyperplane by minimizing the error of a training set. This error is best minimized by a certain equation, this equation can be linear or nonlinear in nature.

In a particular example, each image captured by the thermal sensing device 102 is separated into five smaller images. The number of images could be fewer or greater, however, an odd number of images is desirable. The five smaller images were provided as the input images to the artificial neural network including the modified VGG16 architecture in FIG. 10 and then passed through the PCA and SVM as described above. The SVM takes the data fed from the PCA and considers the output to simplify the data to a single classification entity, e.g., febrile or afebrile, for each of the five smaller images. Since there are an odd number of smaller images, whichever classification is greatest among the five smaller images is output as the classification for the larger image that was captured by the thermal sensing device 102.

Some implementations may include more than one independent relationship or iteration of the above techniques to result in more than one designation. As an example, an implementation may include a classification based upon the relationship of the Left Inner Cantus (LE3), tip of nose (N2), and left side of forehead (F1) along with an independent classification that is based off of RE3, N2, and F3 and another independent classification that looks at LE3 and RE3. Those classification networks would each supply a designation or status (as an example, health or sickness) with the majority designation being relayed to the user. An alternative implementation may include multiple instances of the same classification (as an example, a relationship of LE3, N2, and F1) with inputs subject to different pre-processing. Each classification would provide a designation of status and the majority designation would be relayed to the user.

As discussed above, a febrile classification for a biological subject can be determined based on features in a thermal image of the biological subject. Instead of being predetermined features that were chosen by an individual (e.g., the biological subject's forehead, inner eye corner or auricular meatus), the features are determined by a machine learning process. This may also be in combination with features determined by a person with the person highlighting specific features to be included and the machine learning process applying weights to them (up to and including 0, forcing a feature the person selected to be ignored). As part of the machine learning process, input thermal images of a plurality of febrile biological subjects and afebrile biological subjects were provided as data inputs to the machine learning process and the input thermal images were obtained at (1) various acclimation times, distances or emotional states for the febrile biological subjects and afebrile biological subjects, or (2) at different ambient temperatures.

### III. Other Exemplary Embodiments

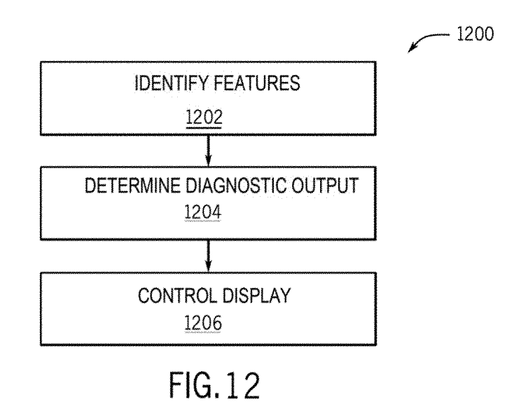

Additional exemplary embodiments implementing the methods and systems discussed above will now be described. As mentioned herein, the methods for biological measurement discussed above with FIGS. 4, 5, 8, and 9 can be implemented with the thermal sensing device 102 of FIG. 1, for example, the non-contact thermometer 200 of FIG. 2. An exemplary biological measurement method 1200 will now be described with reference to FIGS. 2 and 12. However, it is understood, that one or more of the blocks of any method described herein can be implemented with one or more of the blocks of the method 1200. As shown in FIG. 2, the plurality of sensors 112 include the infrared sensor S₁ and the imaging sensor S₂. The infrared sensor S₁ can capture infrared data from the biological subject (e.g., biological being 602) and the imaging sensor S₂ can capture a thermal image of the biological subject. For example, the thermal image 600 of the biological subject 602 of FIG. 6A. In this embodiment, the thermal image includes both eyes of the biological subject.

The display 114 can provide a diagnostic output about the biological subject. For example, as discussed herein, the diagnostic output can be is at least one of a core body temperature of the biological subject, a febrile or non-febrile classification of the biological subject, or a medical diagnosis of the biological subject. In some embodiments, the plurality of sensors 112 can include a visual imaging sensor also operably connected for computer communication with the processor 108. The visual imaging sensors can be offset from the imaging sensor S₂ for capturing an image with a visible light spectrum. Thus, the thermal image includes fewer pixels than the image capturing the visible light spectrum. In this example, corresponding IR data for each physiological structure includes matching the thermal image with the image capturing the visible light spectrum based on the offset between the imaging sensor and the visual imaging sensor.

As shown in FIGS. 1 and 2, the processor 108 is operably connected for computer communication with the plurality of sensors 112 and the display 114. Accordingly, at block 1202 of FIG. 12, the processor 108 can identify at least one feature in the thermal image using a machine learning process. For example, as discussed with block 404 of FIG. 4, areas of interest and/or physiological structures can be identified using, for example, the neural network 118. More specifically, as discussed above with FIG. 5, the plurality of physiological structures can be determined using a segmentation model which identifies areas of interest in the thermal image. In one embodiment, the features include facial features other than the biological subject's forehead, inner eye corner and auricular meatus.

As discussed above with FIGS. 10 and 11, the machine learning process can be trained with input thermal images of a plurality of febrile biological subjects and non-febrile biological subjects. In some embodiments, the input thermal images includes both eyes for each febrile biological subject and afebrile biological subject of the plurality of febrile biological subjects and the plurality of afebrile biological subjects. These input thermal images are obtained at various acclimation times, distances, or emotional states for the febrile biological subjects and the non-febrile biological subjects. Further, the input thermal images can be obtained at different ambient temperatures. More specifically, the input thermal images can be obtained using a thermal imaging camera (e.g., the imaging sensor S₁) at various acclimation times, wherein each acclimation time is an amount of time in which each respective febrile biological subject or afebrile biological subject resides in a controlled temperature environment prior to capturing the respective thermal input image for the respective febrile biological subject or afebrile biological subject in the controlled temperature environment. The input thermal images can also be obtained using a thermal imaging camera at various distances between the thermal imaging camera and the plurality of febrile biological subjects and afebrile biological subjects.

Referring again to FIG. 12, at block 1204, the processor 108 also determines the diagnostic output based on the infrared data corresponding to the at least one feature. For example, the processor 108 can determine a core body temperature based on the feature identified in the thermal images using a regression model from the neural network database 122. Thus, in some embodiments, the method 1200 can determine a febrile state of the biological subject. Further, at block 1206, the processor 108 controls the display 114 to provide the diagnostic output. In one embodiment, the processor 108 controls the display 114 to display the diagnostic output and the thermal image. It is understood that various other embodiments of a thermal sensing device and methods of biological measurement using a neural network can be implemented with the embodiments discussed herein.

### IV. Definitions

The following includes definitions of selected terms employed herein. The definitions include various examples and/or forms of components that fall within the scope of a term and that can be used for implementation. The examples are not intended to be limiting. Further, the components discussed herein, can be combined, omitted or organized with other components or into organized into different architectures.

“Bus,” as used herein, refers to an interconnected architecture that is operably connected to other computer components inside a computer or between computers. The bus can transfer data between the computer components. The bus can be a memory bus, a memory processor, a peripheral bus, an external bus, a crossbar switch, and/or a local bus, among others. The bus can use protocols such as Media Oriented Systems Transport (MOST), Processor Area network (CAN), Local Interconnect network (LIN), among others.

“Component”, as used herein, refers to a computer-related entity (e.g., hardware, firmware, instructions in execution, combinations thereof). Computer components may include, for example, a process running on a processor, a processor, an object, an executable, a thread of execution, and a computer. A computer component(s) can reside within a process and/or thread. A computer component can be localized on one computer and/or can be distributed between multiple computers.

“Computer communication”, as used herein, refers to a communication between two or more computing devices (e.g., computer, personal digital assistant, cellular telephone, network device) and can be, for example, a network transfer, a file transfer, an applet transfer, an email, a hypertext transfer protocol (HTTP) transfer, and so on. A computer communication can occur across, for example, a wireless system (e.g., IEEE 802.11), an Ethernet system (e.g., IEEE 802.3), a token ring system (e.g., IEEE 802.5), a local area network (LAN), a wide area network (WAN), a point-to-point system, a circuit switching system, a packet switching system, among others.

“Computer-readable medium,” as used herein, refers to a non-transitory computer readable storage medium that stores instructions and/or data. A computer-readable medium can take forms, including, but not limited to, non-volatile media, and volatile media. Non-volatile media can include, for example, optical disks, magnetic disks, and so on. Volatile media can include, for example, semiconductor memories, dynamic memory, and so on. Common forms of a computer-readable medium can include, but are not limited to, a floppy disk, a flexible disk, a hard disk, a magnetic tape, other magnetic medium, an ASIC, a CD, other optical medium, a RAM, a ROM, a memory chip or card, a memory stick, and other media from which a computer, a processor or other electronic device can read.

“Diagnostic Output,” as used herein, can include, but is not limited to, any value, classification and/or condition. For example, a core body temperature, a febrile classification, a non-febrile classification, a health condition, a medical diagnosis, among others.

“Database,” as used herein, is used to refer to a table. In other examples, “database” can be used to refer to a set of tables. In still other examples, “database” can refer to a set of data stores and methods for accessing and/or manipulating those data stores. A database can be stored, for example, at a disk and/or a memory.

“Disk,” as used herein can be, for example, a magnetic disk drive, a solid-state disk drive, a floppy disk drive, a tape drive, a Zip drive, a flash memory card, and/or a memory stick. Furthermore, the disk can be a CD-ROM (compact disk ROM), a CD recordable drive (CD-R drive), a CD rewritable drive (CD-RW drive), and/or a digital video ROM drive (DVD ROM). The disk can store an operating system that controls or allocates resources of a computing device.

“Display”, as used herein can include, but is not limited to, LED display panels, LCD display panels, CRT display, plasma display panels, touch screen displays, among others, that can display information. The display can receive input (e.g., touch input, keyboard input, input from various other input devices, etc.) from a user. In some embodiments, the display is part of a portable device (e.g., in possession or associated with a user), a wearable device, a medical device, among others.

“Input/output device” (I/O device) as used herein can include devices for receiving input and/or devices for outputting data. Specifically, the term “input device” includes, but it not limited to: keyboard, microphones, pointing and selection devices, cameras, imaging devices, video cards, displays, push buttons, rotary knobs, and the like. The term “input device” additionally includes graphical input controls that take place within a user interface which can be displayed by various types of mechanisms such as software and hardware based controls, interfaces, touch screens, touch pads or plug and play devices. An “output device” includes, but is not limited to: display devices, and other devices for outputting information and functions.

“Logic circuitry,” as used herein, includes, but is not limited to, hardware, firmware, a non-transitory computer readable medium that stores instructions, instructions in execution on a machine, and/or to cause (e.g., execute) an action(s) from another logic circuitry, module, method and/or system. Logic circuitry can include and/or be a part of a processor controlled by an algorithm, a discrete logic (e.g., ASIC), an analog circuit, a digital circuit, a programmed logic device, a memory device containing instructions, and so on. Logic can include one or more gates, combinations of gates, or other circuit components. Where multiple logics are described, it can be possible to incorporate the multiple logics into one physical logic. Similarly, where a single logic is described, it can be possible to distribute that single logic between multiple physical logics.

“Memory,” as used herein can include volatile memory and/or nonvolatile memory. Non-volatile memory can include, for example, ROM (read only memory), PROM (programmable read only memory), EPROM (erasable PROM), and EEPROM (electrically erasable PROM). Volatile memory can include, for example, RAM (random access memory), synchronous RAM (SRAM), dynamic RAM (DRAM), synchronous DRAM (SDRAM), double data rate SDRAM (DDRSDRAM), and direct RAM bus RAM (DRRAM). The memory can store an operating system that controls or allocates resources of a computing device.

“Operable connection,” or a connection by which entities are “operably connected,” is one in which signals, physical communications, and/or logical communications can be sent and/or received. An operable connection can include a wireless interface, a physical interface, a data interface, and/or an electrical interface.

“Module”, as used herein, includes, but is not limited to, non-transitory computer readable medium that stores instructions, instructions in execution on a machine, hardware, firmware, software in execution on a machine, and/or combinations of each to perform a function(s) or an action(s), and/or to cause a function or action from another module, method, and/or system. A module can also include logic, a software controlled microprocessor, a discrete logic circuit, an analog circuit, a digital circuit, a programmed logic device, a memory device containing executing instructions, logic gates, a combination of gates, and/or other circuit components. Multiple modules can be combined into one module and single modules can be distributed among multiple modules.

“Portable device”, as used herein, is a computing device typically having a display screen with user input (e.g., touch, keyboard) and a processor for computing. Portable devices include, but are not limited to, handheld devices, mobile devices, wearable devices, smart phones, laptops, tablets and e-readers.

“Processor,” as used herein, processes signals and performs general computing and arithmetic functions. Signals processed by the processor can include digital signals, data signals, computer instructions, processor instructions, messages, a bit, a bit stream, that can be received, transmitted and/or detected. Generally, the processor can be a variety of various processors including multiple single and multicore processors and co-processors and other multiple single and multicore processor and co-processor architectures. The processor can include logic circuitry to execute actions and/or algorithms.

It should be apparent from the foregoing description that various exemplary embodiments of the invention may be implemented in hardware. Furthermore, various exemplary embodiments may be implemented as instructions stored on a non-transitory machine-readable storage medium, such as a volatile or non-volatile memory, which may be read and executed by at least one processor to perform the operations described in detail herein. A machine-readable storage medium may include any mechanism for storing information in a form readable by a machine, such as a personal or laptop computer, a server, or other computing device. Thus, a non-transitory machine-readable storage medium excludes transitory signals but may include both volatile and non-volatile memories, including but not limited to read-only memory (ROM), random-access memory (RAM), magnetic disk storage media, optical storage media, flash-memory devices, and similar storage media.

It will be appreciated that various implementations of the above-disclosed and other features and functions, or alternatives or varieties thereof, may be desirably combined into many other different systems or applications. Also that various presently unforeseen or unanticipated alternatives, modifications, variations or improvements therein may be subsequently made by those skilled in the art which are also intended to be encompassed by the following claims.

1. A thermal sensing device, comprising:
a plurality of sensors including at least one of an infrared sensor for capturing infrared data from a biological subject and an imaging sensor for capturing a thermal image of the biological subject;
a display for providing a diagnostic output about the biological subject; and
a processor operably connected for computer communication with the plurality of sensors and the display, wherein the processor:
identifies at least one feature in the thermal image using a machine learning process;
determines the diagnostic output based on the infrared data corresponding to the at least one feature; and
controls the display to provide the diagnostic output.

2. The thermal sensing device of claim 1, wherein the diagnostic output is at least one of a core body temperature of the biological subject, a febrile or non-febrile classification of the biological subject, or a medical diagnosis of the biological subject.

3. The thermal sensing device of claim 1, including a visual imaging sensor operably connected for computer communication to the processor, the visual imaging sensor offset from the imaging sensor for capturing an image with a visible light spectrum.

4. The thermal sensing device of claim 1, wherein the machine learning process includes an artificial neural network and includes a neural network database operably connected for computer communication to the processor, wherein the processor determines the core body temperature based on the feature identified in the thermal image using a regression model from the neural network database.

5. The thermal sensing device of claim 1, wherein input thermal images of a plurality of febrile biological subjects and non-febrile biological subjects were provided as data inputs to the machine learning process and the input thermal images were obtained at (1) various acclimation times, distances or emotional states for the febrile biological subjects and non-febrile biological subjects, or (2) at different ambient temperatures.

6. The thermal sensing device of claim 1, wherein the feature is a plurality of physiological structures determined using a segmentation model which identifies areas of interest in the thermal image.

7. The thermal sensing device of claim 6, including corresponding IR data for each physiological structure by matching the thermal image with the image capturing the visible light spectrum based on the offset between the imaging sensor and the visual imaging sensor.

8. The thermal sensing device of claim 1, wherein the processor controls the display to output the diagnostic output and the thermal image.

9. A computer-implemented method for thermal sensing of a biological subject, comprising:
receiving infrared data about the biological subject from an infrared sensor and a thermal image about the biological subject from an imaging sensor;
identifying at least one feature in the thermal image using a machine learning process;
determining a diagnostic output based on the infrared data corresponding to the at least one feature; and
controlling a display of a thermal sensing device to provide the diagnostic output.

10. The computer-implemented method of claim 9, wherein the diagnostic output is at least one of a core body temperature of the biological subject, a febrile or non-febrile classification of the biological subject, or a medical diagnosis of the biological subject.

11. The computer-implemented method of claim 10, wherein the machine learning process includes determining the core body temperature based on the feature identified in the thermal image using a regression model from a neural network database.

12. The computer-implemented method of claim 11, wherein input thermal images of a plurality of febrile biological subjects and non-febrile biological subjects were provided as data inputs to the machine learning process and the input thermal images were obtained at (1) various acclimation times, distances or emotional states for the febrile biological subjects and afebrile biological subjects, or (2) at different ambient temperatures.

13. The computer-implemented method of claim 9, wherein the feature is a plurality of physiological structures and the method includes determining using a segmentation model areas of interest in the thermal image.

14. The computer-implemented method of claim 9, wherein the at least one image is a facial image that includes both eyes of the biological subject, which is a human being.

15. The computer-implemented method of claim 9, including controlling the display of the thermal sensing device to provide the diagnostic output and the thermal image.

16. A device for biological data measurement, comprising:
a plurality of sensors for measuring biometric data about a biological subject; and
a processor operably connected for computer communication to the plurality of sensors, wherein the processor is operable to receive the biometric data from the plurality of sensors, identify multiple physiological structures of the biological subject, derive biological data associated with each physiological structure of the multiple physiological structures, and determine a diagnostic output based on the biological data associated with each physiological structure of the multiple physiological structures.

17. The device of claim 16, wherein the plurality of sensors includes an infrared sensor for capturing infrared data and an imaging sensor for capturing a thermal image of the biological subject.

18. The device of claim 17, including a display operably connected for computer communication to the processor, wherein the processor controls the display to output the diagnostic output and the thermal image.

19. The device of claim 16, including a neural network database operably connected for computer communication to the processor, wherein the processor determines a core body temperature based on the multiple physiological structures of the biological subject using a regression model from the neural network database.

20. The device of claim 16, wherein the processor identifies a febrile status of the biological subject based on the multiple physiological structures of the biological subject.
